(19) United States
(12) Patent Application Publication
(10) Pub. No.: US 2008/0014215 A1
(43) Pub. Date: Jan. 17, 2008

(54) Animal Bioreactors

(76) Inventor: Frederick R. Blattner, Madison, WI (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 10/558,043

(22) PCT Filed: May 27, 2004
(86) PCT No.: PCT/US04/16657

§ 371 Date: Dec. 28, 2006

Related U.S. Application Data
(60) Provisional application No. 60/473,808, filed on May 27, 2003.
(60) Provisional application No. 60/473,759, filed on May 28, 2003.

(51) Int. Cl. A61K 35/74; A01K 67/027; A61K 39/02; A61P 1/00; C12P 1/00

(52) U.S. Cl. 424/200.1; 424/93.2; 435/41; 800/13; 800/14; 800/15; 800/16; 800/17; 800/18; 800/8

(57) ABSTRACT

The present invention relates to the use of the digestive tract of an animal as a bioreactor for the production of a product of interest.

### FIELD OF THE INVENTION

The invention relates generally to materials and methods for the production of bacterial products using the digestive tract of animals as bioreactors.

### BACKGROUND OF THE INVENTION

Microorganisms are commonly used to produce a wide range of commercial products. For example, Streptomyces strains and Bacillus strains have been used to produce antibiotics. Pseudomonas denitrificans and Propionibacterium strains have been used to produce vitamin B₁₂. Brevibacterium flavum and Corynebacterium glutamicum have been used to produce lysine and glutamic acid, respectively, while other bacteria have been used to produce other amino acids. Although a wide range of both prokaryotic and eukaryotic organisms can express foreign genes, most commercially important products are produced on a large scale in Escherichia coli using fermenters.

Industrial fermenters used for manufacturing products from microorganisms are complex and costly. The design of a fermenter is important to ensure adequate sterility and provide for appropriate levels of containment of the microorganism. The fermenter usually includes probes that permit the accurate and continuous on-line monitoring of as many critical reaction parameters as possible so that adjustments can be made throughout the course of the fermentation process.

A typical large-scale fermentation and product purification procedure begins with preparation and sterilization of the growth medium and sterilization of the fermentation equipment. Cells are typically grown first as a stock culture (5 to 10 ml), then in a shake flask (200 to 1,000 ml), and then in a seed fermenter (10 to 100 liters). Finally, the production fermenter (1,000 to 100,000 liters) is inoculated. After fermentation is complete, the cells are separated from the culture fluid by either centrifugation or filtration. If the product is extracellular, the product is purified directly from the culture medium. If the product is intracellular, the cells are disrupted, the cell debris is removed, and the product is recovered from the debris-free fluid.

Although good growth of aerobic microorganisms can usually be achieved in a standard laboratory flask aerated by a mixer, the industrial production of microorganisms and their products is more complex than simply scaling up bench-scale conditions. Problems associated with the large-scale growth of microorganisms in fermenters include, for example, acetate formation, oxygen supply, mixing efficiency, carbon dioxide and nonequilibrium levels of nutrients. The optimal conditions generally change with each 10-fold increase in the volume of a fermenter. A number of parameters must be precisely regulated to obtain maximum yields from either small (1 to 10 liters) or large (>1,000 liters) cultures. Such parameters include temperature, pH, rate, the nature of mixing of the growing cells, and, with aerobic organisms, oxygen demand.

In view of the cost and complexity associated with producing products in microorganisms, there is a need for more simple and less expensive materials and methods that allow appropriate conditions to be maintained in a range of reaction volumes.

### SUMMARY OF THE INVENTION

The present invention is directed to an animal with a bacteria in the digestive tract of the animal. The bacteria may be a modified bacteria. The animal may be a bird, a reptile or a mammal. The animal may be a cow, pig, goat, sheep, rabbit, horse, mouse, rat or guinea pig. The bacteria may be present in the lumen or the rumen, in the case of a ruminant animal. In one aspect of the invention, the bacteria may comprise a plasmid with a heterologous nucleic acid which may be operatively linked to a regulatory element. In another aspect, the bacteria may comprise an amplified endogenous gene. In another aspect, the bacteria may over-express a bacterial product. In yet another aspect, the bacteria may comprise an inactivated endogenous gene.

The present invention is also directed to a method of producing an animal bioreactor by administering a composition comprising a bacteria to the digestive tract of an animal. The bacteria may be administered to the lumen or the rumen, in the case of a ruminant animal. The bacteria may be introduced to the animal by oral, nasal, or rectal administration and by injection. The animal may be a germ free, gnotobiotic, specific pathogen free or transgenic animal. In one aspect, the bacteria may comprise a plasmid or other vector with a heterologous nucleic acid which may be operatively linked to a regulatory element. In another aspect, the bacteria may comprise an amplified endogenous gene. In another aspect, the bacteria may over-express a bacterial product. In yet another aspect, the bacteria may comprise an inactivated endogenous gene.

The present invention is also directed to a method of producing a product using an animal bioreactor. The bioreactor is an animal with bacteria present in its digestive tract. The bacteria may be present in the lumen or the rumen, in the case of a ruminant animal. In one aspect of the invention, the bacteria may comprise a plasmid with a heterologous nucleic acid that codes for the product or codes for a protein which is involved in the production of the product. In another aspect, the bacteria may comprise an amplified endogenous gene that codes for the product or codes for a protein which is involved in the production of the product. In another aspect, the bacteria may over-express the product. In yet another aspect, the bacteria may comprise an inactivated endogenous gene that normally is involved in the lowering the expression of the product. The product may be isolated from the feces of the animal bioreactor.

### BRIEF DESCRIPTION OF THE DRAWINGS

FIG. 1 shows an enclosure to house mice in comparative microbial isolation.

### DETAILED DESCRIPTION

The present invention relates to the production of a product of interest using animals as bioreactors. The product of interest is produced by a microorganism present in the digestive tract of an animal, preferably the colon. The environment of the digestive tract serves as a bioreactor or fermentation chamber allowing for the growth and maintenance of the microorganism producing the product of interest. The microorganism may then be isolated from the fecal matter.

Substantial cost savings may be achieved using animals as bioreactors because animals are less expensive to purchase and maintain than commercial bioreactors. Furthermore, the maintenance of animal bioreactors is substantially less complex than that of commercial bioreactors.

The present invention also relates to a method of producing an animal bioreactor comprising administering to the digestive tract of a host animal a microorganism that produces a product of interest. The present invention also relates to an animal bioreactor with a microorganism that produces a product of interest that is present in the digestive tract of the animal.

### 1. Product of Interest

The product of interest produced in the practice of the present invention may be one or more of any product naturally produced by the microorganism including, but not limited to, chemicals, amino acids, vitamins, cofactors, growth factors, proteins, nucleic acids and intermediates thereof. The product of interest may also be any product that is not naturally produced by the microorganism including, but not limited to, chemicals, amino acids, vitamins, cofactors, growth factors, heterologous proteins and intermediates thereof. “Protein,” whether recombinant or native, when used to describe a protein produced by a microorganism of the present invention means peptides, polypeptides and proteins as well as fragments, derivatives and fusions thereof.

The product of interest may be intracellularly located in the microorganism. The product of interest may also be secreted into the periplasm of a bacterial microorganism. The product of interest may also be secreted into the digestive tract of the host animal.

### 2. Microorganism

The digestive tract of animals consists of many different microenvironments. The local environment in the digestive tract varies depending on the longitudinal position along the alimentary canal as well as radially from the middle of the lumen out to the lining of the gut. For example, the availability of nutrients from the incomplete digestion and absorption of food as it passes down the canal forms longitudinal as well as radial gradients throughout the digestive tract. In addition, the availability of nutritionally rich compounds secreted in the form of mucous at the gut wall is highest in the region nearest the source of the mucous.

The availability of oxygen in the digestive tract is one of the most important conditions for the growth of microorganisms. Oxygen is available in small amounts from the capillary bed under the intestinal surface whereas the interior of the lumen is strictly anaerobic. Facultative anaerobes such as E. coli in the microaerobic environment located at the gut surface are believed to shield the obligate anaerobes of the lumen from the harmful effects of oxygen by metabolizing the available oxygen.

Other factors that influence the microenvironment of the digestive tract include proximity to different cell types on the gut wall, the relationship to intestinal villae and interactions between different microorganisms in the lumen. As a result of the numerous microenvironments in the gut, several hundred species of microorganisms are able to coexist. Further discussion of the microenvironments of the digestive tract, and microorganism colonization thereof, may be found in Sweeney et al., Infection and Immunity (1996), Allen, A., Physiology of the Gastrointestinal Tract, Raven Press, New York (1981), and Carlstedt-Duke, B., The regulatory and protective role of the normal microflora. The Macmillan Press Ltd., London (1989), the contents of which are hereby incorporated by reference in their entirety.

The microorganism used in the practice of the invention may be one or more of any microorganisms that are capable of being maintained in the digestive tract of the host animal, and in particular the colon or rumen. The microorganisms may be a bacteria, virus, fungi, or yeast. The microorganism may be an aerobic, facultative anaerobic, or anaerobic organism. Aerobic microorganisms may be microaerophillic. The microorganism may produce the product of interest in one or all of the available microenvironments of the alimentary canal.

The microorganism may be present in the lumen of a host animal. The microorganism may also be present in the rumen of a ruminant host animal. Representative microorganisms useful in the practice of the present invention are available from the American Type Culture Collection. Other representative microorganisms are described in S. Y. Lee, “High Density Culture of Escherichia coli,” Tibtech 14:98-103 (1996), the contents of which are hereby incorporated by reference in their entirety.

a. Wild Type Microorganisms

The microorganism may be any wild-type or native strain that is not naturally present in the digestive tract of the host animal. The microorganism may also be a strain that over-expresses the product of interest.

b. Modified Microorganisms

1) Native and Foreign Products of Interest
a) Over-expression of Native Products

The microorganism may also be modified to over-express a product of interest that is native to the microorganism. A native product of interest may be over-expressed by amplification of a gene native to the microorganism. A native product of interest may also be over-expressed by inactivating a gene native to the microorganism that normally represses production of the product of interest or degrades the product of interest. A native product of interest may also be over-expressed by introducing a heterologous gene to the microorganism that allows for increased levels of the microorganism to be present in the digestive tract of the host animal. For purposes of modified microorganisms, references to “gene” also encompass more than one gene. For example, a microorganism may be modified to add or delete genes from a metabolic or signaling pathway.
b) Expression of Foreign Products

The microorganism may also be modified to produce a product of interest that is foreign to the microorganism. For example, the microorganism may be modified by recombinant DNA techniques well known in the art to contain a foreign gene that may be expressed to produce a recombinant protein. The recombinant protein may be a product of interest. In addition the recombinant protein may regulate or catalyze production of a product of interest that is foreign to the microorganism. “Expression” and “over-expression” when used to describe the production of a product of interest encompass proteinaceous products as well as the product, substrate or intermediate of a catalytic protein.

2) Modification

A gene may be introduced, amplified, or inactivated by using methods well known in the art which are described in Sambrook et al, “Molecular Cloning Manual,” Cold Spring Harbor Laboratory (2001), the contents of which are hereby incorporated by reference in their entirety.
a) Vectors

Heterologous genes may be introduced to a microorganism using a vector comprising the foreign gene. The vector may be capable of autonomous replication or it may integrate into the DNA of the microorganism. As used herein, the term “vector” refers to a nucleic acid molecule capable of transporting another nucleic acid to which it has been linked including, but not limited to, a plasmid, cosmid, phage, phagemid, YAC or viral vector.

The vector may be in a form suitable for expression of the heterologous gene in the microorganism. The expression vectors may be designed for expression of recombinant protein products in prokaryotic or eukaryotic cells. Suitable host cells are discussed further in Goeddel, Gene Expression Technology: Methods in Enzymology 185, Academic Press, San Diego, Calif. (1990), the contents of which are hereby incorporated in their entirety. Methods for the optimization of protein expression in microorganisms are discussed further in Hannig et al., Trends Biotechnol., 16(2):54-60 (1998), the contents of which are hereby incorporated by reference in their entirety.
b) Regulated Expression

The microorganism may constitutively express the heterologous gene. Expression of the heterologous gene may also be regulated by one or more operatively linked regulatory sequences. The term “regulatory sequence” includes promoters, enhancers and other expression control elements (e.g., polyadenylation signals). Regulatory sequences also include those sequences that direct constitutive expression of a nucleotide sequence, as well as tissue-specific regulatory and/or inducible sequences. Inducible regulatory sequences may be activated or repressed based on the local environment of the microorganism in the host. Inducible regulatory sequences include, but are not limited to, control sequences for anaerobiosis or aerobiosis. The effect of local environment on gene expression is discussed further in Cheung et al., J. Bacteriol. (1993), the contents of which are hereby incorporated by reference in their entirety.

Control systems for expression in aerobic microenvironments include the lac, pho, T7 and arabinose expression systems. In addition, promoters may be used that are functionally more active under aerobic conditions. Exemplary promoters for expression under aerobic conditions are listed in Table 1. Production of the product of interest in anaerobic microenvironments may use promoters that are more active under anaerobic conditions including, but not limited to, the promoters listed in Table 2. The promoters for cyoA and cyoD induce transcription of subunits of cytochrome ubiquinol oxidase in the absence of oxygen but are quiescent in aerobically growing cells. The promoter for ydfZ, a gene of E. coli whose function at present is not known, is strongly expressed in the presence of oxygen but is repressed under anaerobic conditions. The promoters for glpA and glpB show similar regulation in the presence of oxygen.

TABLE 1

| | | Genes whose expression change between anaerobic and areobic | | | | | | |
|---|---|---|---|---|---|---|---|---|
| | | conditions ranks in the top 200 and whose expression level is in | | | m-RNA | m-RNA | Rank of | Rank of |
| | | the top 400 under at least one of the conditions | | | Copies per | Copies per | Anareobic | Aerobic |
| | | strongly expressed m-RNAs relatively strongly expressed m-RNAs | Fold | Rank of | Anaerobic | Aerobic | m- | m-RNA |
| bnum | gene | relatively SHUT DOWN in anaerobically growing cells | change | change | cell | cell | RNA level | level |
| b0432 | cyoA | cytochrome o ubiquinol oxidase subunit II Aerobic respiration | 146.8 | 1 | 0.11 | 16.81 | 3942 | 164 |
| b0429 | cyoD | cytochrome o ubiquinol oxidase subunit IV/Aerobic rewspiration | 95.3 | 2 | 0.12 | 11.60 | 3921 | 247 |
| b0722 | sdhD | succinate dehydrogenase, hydrophobic subunit/TCA cycle | 36.6 | 5 | 0.23 | 8.56 | 3571 | 320 |
| b0431 | cyoB | cytochrome o ubiquinol oxidase subunit I/Aerobic respiration | 38.1 | 7 | 0.43 | 16.32 | 3041 | 170 |
| b0721 | sdhC | succinate dehydrogenase, cytochrome b556/TCA cycle | 39.7 | 8 | 0.20 | 8.04 | 3875 | 334 |
| b3908 | sodA | superoxide dismutase, manganese/Detoxification | 32.7 | 10 | 0.51 | 16.52 | 2842 | 167 |
| b0430 | cyoC | cytochrome o ubiquinol oxidase subunit III/Anerobic respiration | 25.2 | 15 | 0.50 | 12.70 | 2843 | 221 |
| b2518 | ndk | nucleoside diphosphate kinase/Purtne ribonucleotide biosynthesis | 11.2 | 29 | 1.10 | 12.37 | 1867 | 230 |
| b0723 | sdhA | succinate dehydrogenase, catalytic and NAD/flavoprotein | 10.3 | 34 | 0.95 | 9.82 | 2028 | 285 |
| | | subunit/TCA cycle | | | | | | |
| b0725 | (none) | orf. hypothetical protein, unknown CDS/Unknown function | 7.2 | 54 | 0.91 | 6.58 | 2091 | 390 |
| b3238 | mdh | malate dehydrogenase, NAD(P)-binding/TCA cycle | 6.5 | 58 | 3.09 | 19.95 | 799 | 139 |
| b0728 | sucC | succinyl-CoA synthetase, beta subunit/TCA cycle | 6.0 | 73 | 2.64 | 15.77 | 914 | 176 |
| b2587 | kgtP | alpha-ketoglutarate permease (MFS family)/Transport of small | 5.4 | 83 | 1.68 | 9.04 | 1358 | 308 |
| | | molecules: Carbohydrates, organic acids, alcohols | | | | | | |
| b0727 | sucB | 2-oxoglutarate dehydrogenase-dihydrolipoyltranssuccinate | 6.2 | 84 | 1.86 | 11.47 | 1260 | 249 |
| | | transferase, E2 component of the enzyme complex/TCA cycle | | | | | | |
| b0726 | sucA | 2-oxoglutarate dehydrogenase (thiamin-binding decarboxylase | 5.7 | 98 | 1.22 | 7.01 | 1723 | 367 |
| | | component of the enzyme complex)/TCA cycle | | | | | | |
| b1136 | icdA | isocitrate dehydrogenase, specific for NADP + on s14 prophage;/ | 4.9 | 106 | 6.94 | 33.70 | 369 | 83 |
| | | TCA cycle | | | | | | |
| b0720 | gltA | citrate synthase/TCA cycle | 4.2 | 138 | 2.60 | 10.96 | 938 | 259 |
| b4015 | aceA | isocitrate lyase/Central intermediary metabolism: Glyoxylate | 3.9 | 154 | 2.01 | 7.82 | 1178 | 339 |
| | | bypass | | | | | | |
| b0126 | yadF | putative carbonic anhdrase (EC 4.2.1.1)/not classified | 3.5 | 160 | 5.83 | 20.40 | 435 | 137 |

TABLE 2

| | | Genes whose expression change between anaerobic and areobic | | | | m-RNA | | |
|---|---|---|---|---|---|---|---|---|
| | | conditions ranks in the top 200 and whose expression level is | | | m-RNA | Copies | Rank of | Rank of |
| | | in the top 400 under at least one of the conditions | | | Copies per | per | Anareobic | Aerobic |
| | | strongly expressed m-RNAs relatively TURNED | Fold | Rank of | Anaerobic | Aerobic | m- | m-RNA |
| bnum | gene | ON in anaerobically growing cells | change | change | cell | cell | RNA level | level |
| b1541 | vdlZ | orf. hypothetical protein./Unknown function | 43.1 | 4 | 62.09 | 1.44 | 14 | 1481 |
| b2242 | glpB | sn-glycerol-3-phosphate dehydrogenase (anaerobic), | 9.2 | 33 | 17.11 | 1.88 | 176 | 1204 |
| | | membrane anchor subunit and FAD/NAD(P)- | | | | | | |
| | | binding domain/Anaerobic respiration | | | | | | |
| b2241 | gtpA | sn-glycerol-3-phosphate dehydrogenase (anaerobic), large subunit/ | 8.0 | 40 | 8.40 | 1.05 | 316 | 1826 |
| | | Anaerobic respiration | | | | | | |
| b3510 | hdeA | orf, hypothetical protein, conserved protein/Unknown function | 6.8 | 50 | 14.17 | 2.10 | 218 | 1087 |
| b2243 | gtpC | sn-glycerol-3-phosphate dehydrogenase (anaerobic), K-small subunit/ | 5.7 | 62 | 6.38 | 1.12 | 397 | 1757 |
| | | Anaerobic respiration | | | | | | |
| b2579 | yllD | putative formate acetyltransferase/Anaerobic respitation | 5.4 | 81 | 16.16 | 3.01 | 183 | 802 |
| b1241 | adhE | multifunctional protein: pyruvate-formate lyase deactivase (N-terminal: | 4.4 | 123 | 24.85 | 5.70 | 123 | 444 |
| | | acetaldehyde-CoA dehydrogenase) (C-terminal: Iron-dependent alcohol | | | | | | |
| | | dehydrogenase)/termentation | | | | | | |
| b0904 | focA | formate transport protein (formate channel 1) (FNT family)/ | 3.4 | 176 | 12.80 | 3.82 | 239 | 654 |
| b1685 | ydiH | orf, hypothetical protein, unknown CDS/Unknown function | 3.2 | 187 | 6.72 | 2.09 | 383 | 1093 |
| b2296 | ackA | acetate kinase A (propionate kinase 2)/Electron transport | 3.3 | 188 | 18.15 | 5.55 | 165 | 453 |

Control regions may be chosen to regulate production of the product of interest in accordance with the desired timing and spatial plan for expression. For example, the use of an inducible promoter may be of use in those circumstances where expression of the product of interest is detrimental to the host animal or the microorganism. In such circumstances, the modified microorganism may be administered to the host animal and allowed to expand to optimum levels in the digestive tract. Once optimum levels of the microorganism have been obtained, an inducing agent may be administered to the host animal. Upon administration of the inducing agent and uptake by the modified microorganism, expression of the product of interest may be activated. Once production of the product of interest has been induced, the fecal matter may be collected by any method including, but not limited to, catheter, enema, surgery or sacrificing of the host animal. In order to protect the host animal from a potentially harmful product of interest, the product of interest may also be sequestered in the cytoplasm of the microorganism or bound to the cell membrane of the microorganism. A product of interest may also be produced in an inactive form. For example, insulin could be produced as pro-insulin. Production of the product of interest may also be limited to microenvironmental niches including, but not limited to, the anaerobic center of the lumen. Alternatively, production of the product of interest may not occur until after the expulsion of fecal matter from the host animal. Once expelled from the host animal, the product of interest may be produced by secondary fermentation or processing. In addition, the production of the product of interest may be under control of an aerobic promoter that is upregulated as fecal matter is expelled into aerobic conditions. Additional examples of inducible expression systems are disclosed in Sambrook et al.
c) Fusion Proteins

Fusion proteins may be produced using an expression vector that adds a number of amino acids to a recombinant protein product, usually to the amino terminus of the recombinant protein. Typical fusion expression vectors include pGEX (Pharmacia Biotech, Inc.; Smith D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A. Fusion vectors may increase production of the product of interest, increase the solubility of the product of interest, or aid in the purification of the product of interest by acting as a ligand in affinity purification. A proteolytic cleavage site may be introduced at the fusion junction for subsequent removal of the fusion moiety after purification of the fusion protein. The proteolytic cleavage site may be a recognition sequences for enzymes including, but not limited to, Factor Xa, thrombin and enterokinase.
d) Selection

The microorganism may also be modified to contain a gene for a selectable marker including, but not limited to, a gene conferring resistance to an antibiotic. For example, a gene encoding beta lactamase which degrades ampicillin may be inserted into the genome of a modified bacteria or added to a vector used for producing the modified bacteria. By feeding the host animal ampicillin in its diet, the modified bacteria may be maintained at the exclusion of other bacteria.
e) Reduced Genome

The microorganism may also be modified to have a reduced genome. Much of the genetic information contained within the genome of a microorganism may be deleted without detrimentally affecting production of the product of interest. Moreover, microorganisms with a reduced genome may be advantageous over native strains in the production of many products of interest. For example. a reduced genome may lead to the production of fewer native products or lower levels thereof which may lead to less complex purification of the product of interest. In addition, microorganisms with a reduced genome may be less metabolically demanding and thus may produce the product of interest more efficiently. The preparation of bacteria with reduced genomes is discussed in copending U.S. patent application Ser. No. 10/057,582, the contents of which are hereby incorporated by reference in their entirety.

### 3. Animals

Any animal with a digestive tract that is capable of supporting an enteric microorganism may be used in the practice of the present invention. The animal may be a bird, reptile or mammal. Any mammal may be used in the practice of the present invention, including, but not limited to, cows, pigs, goats, sheep, rabbits, horses, mice, rats and guinea pigs. The use of animals allows for low cost maintenance and propagation of the animal bioreactors using techniques well known in the area of animal husbandry.
a. Germ Free, Gnotobiotic and Specific Pathogen Free Animals

Germ free (GF), gnotobiotic (GN), or specific pathogen free (SPF) animals may also be used in the practice of the present invention. GF animals may be produced from caesarian-derived stock and maintained in quarters free of animal pathogens. Alternatively, GF animals may be produced by administering antibiotics or other suitable chemotherapeutics to sterilize the digestive tract of the subject animal. The production and maintenance of GN animals is similar to GF animals, except that GN animals have a defined flora. SPF animals also have defined flora as they are modified to be germ free for a particular pathogen. Procedures for preparing and maintaining GF, GN and SPF animals are described by J. R. Pleasants “Gnotobiotics,” Handbook of Laboratory Animal Sciences, p. 119-174 and by G. G. Gomez in “The Colostrum-Deprived, Artificially-Reared, Neonatal Pig As A Model Animal For Studying Rotavirus Gastroenteritis,” Frontiers in Biosciences, 2, d471-481 (1997), the contents of which are hereby incorporated by reference in their entirety.

GF, GN and SPF host animals may be useful for producing and maintaining the desired levels of microorganisms in the digestive tract of the animal fermentation chamber. As described by Baum et al. in “Effect of Feeding Lactobacillus to Pigs Infected with Salmonella typhimurium,” ASL-R 687, Food Safety, Iowa State University, the contents of which are hereby incorporated by reference in its entirety, administering a strain of Lactobacillus to 4 week old pigs as a probiotic results in the elimination of S. typhimurium detectable in the feces. When GF host animals are used, the large intestines may be recolonized with the microorganism. When using GN or SPF host animals, the microorganism may be colonized with the defined flora in the large intestines of the host animals.

When GF, GN or SPF host animals are used, the yield of the product of interest may be increased by housing the animals in sterile conditions which may prevent recolonization by other strains of microorganisms. It may also be possible to maintain acceptable yields of a product of interest using GF, GN or SPF animals without maintaining the host animals in sterile conditions.
b. Transgenic Animals

The host animal may also be a transgenic animal, the production of which is well known in the art. A transgenic host animal may contain a foreign gene encoding a protein that when expressed in the transgenic host may be beneficial for reasons including, but not limited to, increased production of the product of interest, modification of the product of interest or a precursor thereto, or increased viability of the microorganism in the digestive tract of the host animal. For example, a transgenic pig expressing the enzyme phytase in its salivary glands in order to use an alternative source of phosphorous is described by C. W. Forsberg in “The Enviropig an Environmentally Friendly Pig That Utilizes Plant Phosphorous More Efficiently,” World Business Newspaper (2001)), the contents of which are hereby incorporated by reference in its entirety.

### 4. Administration

Animal bioreactors may be produced by administering a microorganism to the host animal by any method which allows for introduction of the microorganism to the digestive tract of the host animal including, but not limited to, oral, nasal, and rectal administration. The microorganism may be formulated in any manner which allows for introduction and propagation of the microorganism in the digestive tract of the host animal including, but not limited to liquid cultures, lyophilized cultures, encapsulated cultures, and agar. Procedures for formulating bacterial cultures for administration to cattle and sheep are described by Ware et al. in U.S. Pat. No. 5,534,271, the contents of which are hereby incorporated by reference in their entirety. Procedures for formulating bacterial cultures for administration to pigs are described by Baum et al. Depending on the desired level of modified microorganism in the digestive tract or the desired amount of the product of interest to be produced, the modified microorganism may be administered once and thereafter maintained or repeatedly administered on a periodic basis.

### 5. Isolation of the Product of Interest

The excreted feces may be collected in any suitable manner including, but not limited to, shoveling. Other methods of collecting the product of interest include the use of tubes or catheters sampling a particular microenvironment. The product of interest may be collected under any desired environment, such as anaerobic conditions. Other methods for collecting the product of interest include the use of surgical procedures to remove the intestinal tract or portions thereof. Special housing systems for the animal fermentation chambers may also be used to efficiently collect the excreted feces. van Kempen, North Carolina State University (2002), the contents of which are hereby incorporated in their entirety, describes a system using a floor scraping machine to collect feces from housed animals. Kaspers et al., North Carolina State University Annual Swine Report (2002), the contents of which are hereby incorporated in their entirety, describes a system using an automated conveyor belt for collecting feces from pigs. In view of fecal material being highly compacted with most of the water absorbed in the large intestines, there may not be a need to perform an initial centrifugation step as in traditional fermentation. The collected feces may be treated in a suitable manner to preserve the modified microorganism in the feces as well as preserve the product of interest.

In view of the bulk of the fecal matter being microorganisms, the product of interest may be isolated from the feces using standard purification techniques in the art. If the product of interest is in the feces (culture medium), the feces may be solubilized and the product of interest concentrated from the supernatant followed by purification using standard procedures including, but not limited to, chromatography, centrifugation and extraction, which are described in Janson et al., “Protein Purification: Principles, High-Resolution Methods, and Applications,” Wiley (1998), the contents of which are hereby incorporated by reference in their entirety.

If the product of interest is in the cellular fraction, the microorganisms may be lysed. A large number of chemical, biological and physical methods are known in the art for disrupting microbial cells. Chemical methods for disrupting microbial cell walls include, but are not limited to, treatment with alkali, organic solvents or detergents. If the product of interest is stable at about pH 10.5-12.5, lysis may be carried out on a large scale at low cost. Lysis may also be performed using enzymatic treatments which may be highly specific and which may be performed under mild conditions. The microorganisms may also be physically disrupted by nonmechanical methods including, but not limited to, osmotic shock and repeated cycles of freezing and thawing. Lysis may also be performed by mechanical procedures including, but not limited to, sonication, wet milling, high-pressure homogenization and impingement. Alternatively, lysis may be accomplished by using nematode worms to feast on the fecal matter. Nematode worms including, but not limited to, C. elegans eat fecal matter voraciously and have a grinder organ in the mouth that efficiently pulverizes E. coli. If necessary, the nematode worms may be genetically modified, for example, to avoid digesting the product of interest. One of ordinary skill in the art will be able to select an appropriate method for cell disruption.

After cell disruption, cell debris is removed by methods including, but not limited to, low-speed, high-capacity centrifugation or membrane microfiltration. The product of interest may optionally be concentrated from either the crude lysate, the clarified lysate or the cell-free fecal material using standard techniques including, but not limited to, precipitation with organic solvents or ammonium sulfate and cross-flow ultrafiltration.

A number of proteinaceous products of interest may be present as insoluble particles present in inclusion bodies within the microorganism. After cell disruption, such inclusion bodies may be separated from the bulk of the remaining cell components. The product of interest may be renatured using standard techniques including, but not limited, dissolving in 6 M guanidinium chloride followed by renaturing in an appropriate buffer, as described by Jansen et al.

The required degree of purity of the final product of interest depends on its end use. Crude preparations may be satisfactory in some instances, but additional purification steps may be required for other products of interest using standard separation techniques known in the art including, but not limited to, chromatography, centrifugation and extraction.

### 6. Expression of a Product of Interest to Effect the Host Organism

One of ordinary skill in the art will appreciate that the materials and methods described above may also be used for the direct expression of any product that is otherwise commercially produced in microorganisms. The host animal may be any of the animals described above, as well as a human. Expression of the product of interest by the modified microorganism may have a beneficial effect on the host animal. Examples of products of interest that may be expressed by microorganisms for the benefit of the host animal are described in “High cell-density culture of Escherichia coli,” TIBTECH, March, 98-103 (1996).

A product of interest may be expressed that prevents, represses or treats a disease of the host animal. For example, antigens may be expressed by the modified microorganism as a means of inducing an immune response in the host animal against a specific pathogen, and in particular a mucosal immune response. Antibodies themselves may also be expressed by the modified microorganism.

As an alternative to genetically engineering animals to express proteins encoded by endogenous or heterologous genes, the proteins may be expressed by a modified microorganism in the digestive tract of the animal. For example, cows maybe administered a modified microorganism that expresses bovine growth hormone, which may lead to increased milk production or muscle mass.

Having now generally described the invention, other aspects and advantages of the present invention will be understood upon consideration of the following illustrative examples. These examples are for purposes of illustration only and are not intended to limit the scope of the invention as set out in the appended claims. Example 1 describes the production of germ-free pigs. Example 2 describes the production of bovine growth hormone using a porcine bioreactor. Example 3 described the production of lysine using a porcine bioreactor. Example 4 describes the treatment of humans with gastroenterological viruses.

### EXAMPLE 1

### Production Of Germ-Free Pigs

Pregnant sows are transferred to an isolated farrowing facility five days before farrowing so that germ-free neonatal pigs are farrowed in an antiseptically clean stall after repeated bathing and sanitizing of sows with an iodinated detergent. Sows are attendant-farrowed so that pigs are caught as they are being born, the umbilical cord is clamped with a vale cord clamp to help prevent navel infection, and gently detached from the sow. Each pig is immediately transferred to a clean plastic container, sprayed with a disinfectant solution, freed from adhering membranes and dried with disposable paper towels, and transferred to an isolation room where the umbilical cord is cut and disinfected with a tincture of iodine. Each pig is kept warm with a heating lamp in a disinfected cage until the end of farrowing. Each germ-free pig is then transferred into a separate room containing an automated feeding device.

### EXAMPLE 2

### Production Of Bovine Growth Hormone Using A Porcine Bioreactor

Ten 4-week old germ-free pigs as described in Example 1, which test negative for bacteria by rectal swaps, are fed a 24 hour culture of 10¹⁰ c.f.u. of bacteria previously transformed with pBGH33-4 (U.S. Pat. No. 5,489,529, the contents of which are incorporated in their entirety), which expresses high levels of bovine growth hormone. Rectal swaps from each pig are collected on days 2, 4, 7, 9, 11, 14, 17, 19, 21 and 24 relative to challenge and plated on LB supplemented with tetracycline to measure the growth of the modified bacteria in the large intestines. The rectal swabs are also plated on LB to ensure that other bacteria have not colonized the large intestines.

Feces are collected from the pigs beginning 4 days after induction. The feces are dissolved in Tris buffer and sonicated to lyse the modified bacteria. Bovine growth hormone in the resulting supernatant is then precipitated with 30% to 70% ammonium sulfate and then resuspended and dialyzed in Tris buffer. The dialyzed sample is then loaded on an SDS-polyacrylamide gel. α-BGH antibodies used to probe the gel verify the presence of BGH.

### EXAMPLE 3

### Production Of Threonine Using A Porcine Bioreactor

Ten 4-week old germ-free pigs as described in Example 1, which test negative for bacteria by rectal swaps, are fed a 24 hour culture of 10¹⁰ c.f.u. of a Corynebacterium glutamicum strain that over-expresses L-threonine, as described by Sahm et al., Ann NY Acad Sci 15;782:25-39 (1996), the contents of which are incorporated by reference in their entirety. The C. glutamicum strain is the result of the amplification of the feedback inhibition insensitive homoserine dehydrogenase and homoserine kinase in an L-lysine over-expressing strain, thereby channeling carbon flow from the intermediate aspartate semialdehyde towards homoserine, resulting in a high accumulation of L-threonine.

Rectal swaps from each pig are collected on days 2, 4, 7, 9, 11, 14, 17, 19, 21 and 24 relative to challenge and plated on LB supplemented with tetracycline to measure the growth of the modified bacteria in the large intestines. The rectal swabs are also plated on LB to ensure that other bacteria have not colonized the large intestines.

Feces are collected from the pigs beginning 4 days after induction. The feces are dissolved in Tris buffer and sonicated to lyse the modified bacteria. L-threonine in the resulting supernatant is then purified by batch chromatography as described by Jansen et al.

### EXAMPLE 4

### Treatment of Gastroenterological Viruses

An animal diagnosed with a gastroenterological virus is administered an effective amount of a formulation comprising a bacteria which has been modified to express and secrete a humanized single-chain antibody that is specific for a coat protein of the virus. Methods of producing and secreting human single-chain antibodies are described in U.S. Pat. No. 5,885,793, the contents of which are hereby incorporated by reference. The bacteria is also modified to have expression of an essential bacterial gene placed under the control of a promoter which is regulated by a compound which may be administered to the patient orally, rectally or by i.v. administration.

The administration of the bacterial formulation may take place by any suitable route of administration, including oral, nasal, rectal or by injection. Following the initial administration of the formulation, the patient is periodically administered an effective amount of the compound which induces expression of the essential gene of the bacteria thereby allowing establishment and maintenance of the modified bacteria in the colon. After the initial administration, it is possible that booster doses of the bacterial formulation may be required to obtain optimal bacterial levels in the colon.

Once established in the colon of the patient, the modified bacteria secretes single-chain antibodies specific for a coat protein of the virus, thereby allowing reduction of the number of infectious virus particles. During the course of treatment, the levels of modified bacteria, secreted antibody and viral particles present in the colon may be monitored by assaying fecal samples in order to evaluate the effectiveness of the agent. Once the patient is no longer presenting clinical symptoms and the levels of virus particles in the colon have reduced to acceptable levels, the patient is no longer administered the compound necessary for expression of the essential bacterial gene, thereby allowing elimination of the modified bacteria from the colon of the patient.

### EXAMPLE 5

### Production Of Red Fluorescent Protein Using A Murine Bioreactor

Four male albino mice were purchased from a retail establishment and housed at a private facility. The mice were intubated with 25 mg of streptomycin sulphate in 200 μl of water. The mice were then colonized by being fed for 48 hrs from a feeding bottle containing 100 ml of Luria broth suplimented with 200 μg/ml of ampicillin that had been seeded with 1 ml of an overnight broth culture of DH10B (pDsRed), which is E. coli strain DH10B containing the plasmid pDsRed (CLONTECH). The plasmid pDsRed is derived from the PUC plasmid vector and comprises a Lac promoter driving a red fluorescent protein gene and ampicillin resistance as a selectable marker. During the 48-hour exposure period, the mice frequently drank from this culture as the E. coli containing the plasmid slowly grew to slight turbidity at room temperature with no aeration. Two additional male albino mice were not colonized and served as controls.

At the end of the 48-hour period, the culture was replaced with water containing 200 ug/ml of ampicillin (AMP) and 1 mM IPTG (Isophenylthiogalactaside) to induce expression of the red fluorescent protein. From this point forward the experimental mice were watered with the AMP/IPTG solution while control mice were given sterilized water.

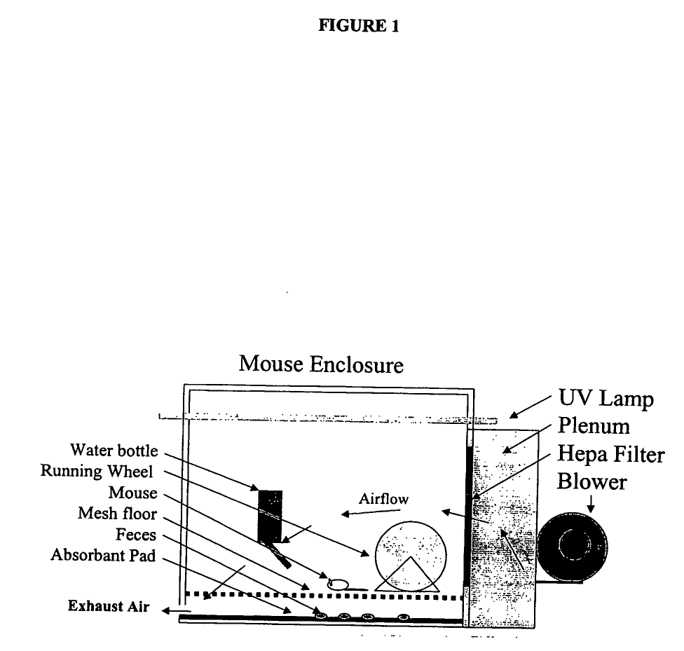

The mice were housed in a sealed plastic enclosure as shown in FIG. 1. The mice were provided with hepa-filtered air at positive pressure. A ½ inch wire mesh platform kept the mice 1″ above an absorbent fecal collection paper which could be slid out through the air exhaust port to collect fecal deposits (pellets). A germicidal lamp in the enclosure was used to sterilize the interior for 30 min approximately once per day during which time the mice were housed temporarily in a sterilized plastic bottle. All animals were fed sterilized pelletized mouse ration. Food and water fed to the mice were sterilized in an autoclave. The control mice were housed in a separate, non-sterile cage and fed the same diet. The mice appeared healthy and exercised frequently on the running wheel provided in the cage.

Fresh fecal deposits were collected from the animals either 2 or 7 days after colonization, then suspended in 1 ml of 0.01 M Tris buffer (pH 7.9) per fecal deposit (ca 2×5 mm pellets). The samples were then serially diluted and plated on LB containing 100 μg/ml ampicillin. Plates were incubated for 48 hrs and then counted. The plates from the experimental animals contained colonies with the pronounced red color characteristic of DH10B (pDsRed) colonies. The red colonies and the results in Table 3 show that the experimental mice were successfully colonized by DH10B (pDsRed).

TABLE 3

| Animal | Cell Counts/ml |
|---|---|
| 1 | 3.8 × 10⁸ |
| 2 | 3.1 × 10⁸ |
| 3 | 7.5 × 10⁷ |
| 4 | 2.0 × 10⁷ |
| Control-1 | no colonies |
| Control-2 | no colonies |

For fluorescence assay, 1 ml of suspended fecal cells, (i.e., one fecal deposit) from the experimental and control animals was centrifuged and washed three times in 0.01 M Tris buffer. As an additional control, broth grown E. coli DH10B cells sufficient to yield a similar sized cell pellet was prepared with the same protocol. The centrifugation pellets were then resuspended in 1 ml of 300 mM NaCl, 50 mM phosphate buffer pH 7.5. 150 μl of the resulting solution was added to 750 μl of 100 mM NaCl, 20 mM phosphate pH 7.5 plus 100 μl “Popculture” lysis solution from Novagen and then sonicated for 5 sec using a 350 watt sonicator at setting 5. The solution was then centrifuged for 10 min at 13000×g in a micro centrifuge. 70 ul (10%) of the cleared supernatant was then assayed in a Molecular Dynamics fluorescence spectrophotometer with excitation wavelength 544 nm and readout wavelength of 590 nm for detection of red fluorescent protein. Fluorescence units were corrected vs a water control showing negligible fluorescence signal. Protein concentration was measured with a Bradford kit from Biorad. The results in Table 4 show that the experimental mice were able to produce red fluorescent protein.

TABLE 4

| | Protein | |
|---|---|---|
| | Concentration | Relative |
| Animal | (mg/ml) | Fluorescence |
| 1 | 0.52 | 69.0 |
| 2 | 0.57 | 52.0 |
| 3 | nd | 33.0 |
| 4 | nd | 15.0 |
| Control-1 | nd | 6.0 |
| Control-2 | nd | 1.7 |
| DH10B cells | 0.54 | 6.0 |

### EXAMPLE 6

### Large-Scale Production Of Threonine Using Bovine Bioreactor

3000 dairy cows are colonized with a bacterial strain that over-expresses L-threonine as described in Example 3. The cows are housed in a dairy facility under comparative microbial isolation. The cows are born in the facility and leave the facility only to be milked. The food rations for the cows is controlled to minimize toxic substances excreted in the feces. The feces are collected via drains in the floor of the facility and transported to a collector on a lower level. Once the feces are collected, L-threonine is isolated as described in Example 3.

The collection system is similar to systems at new generation dairy farms that collect the feces and conduct the fecal matter to a digester that produces methane used to generate power for the facility and the surrounding area. Screen wire is used to keep birds and other animal out of the feces collection system.

The microbial content of the entire herd becomes similar as the animal interact. The microbial content of the herd can also be controlled to maximize production of the L-threonine without interfering with the quality and safety of the milk.

What is claimed is:

1. An animal whose digestive tract contains a modified bacteria.

2. The animal of claim 1, wherein the modified bacteria comprises a plasmid, wherein said plasmid comprises an optionally inducible promoter operatively linked to a heterologous nucleic acid.

3. The animal of claim 1, wherein the modified bacteria comprises an amplified endogenous gene.

4. The animal of claim 3, wherein the endogenous gene is inactivated by mutation, insertion or deletion.

5. The animal of claim 1, wherein the modified bacteria is capable of over-expressing a bacterial product.

6. The animal of claim 1, wherein the modified bacteria comprises an inactivated endogenous gene.

7. The animal according to any one of claims 1-6, wherein said animal is selected from the group consisting of a cow, pig, goat, sheep, rabbit, horse, mouse, rat and guinea pig.

8. A method of producing an animal bioreactor comprising administering to the digestive tract of an animal a composition comprising a modified bacteria.

9. The method of claim 8, wherein the modified bacteria comprises a plasmid, wherein said plasmid comprises an optionally inducible promoter operatively linked to a heterologous nucleic acid.

10. The method of claim 8, wherein the modified bacteria comprises an amplified endogenous gene.

11. The method of claim 10, wherein the endogenous gene is inactivated by mutation, insertion or deletion.

12. The method of claim 8, wherein the modified bacteria is capable of over-expressing a bacterial product.

13. The method of claim 8, wherein the modified bacteria comprises an inactivated endogenous gene.

14. The animal according to any one of claims 8-13, wherein said animal is selected from the group consisting of a cow, pig, goat, sheep, rabbit, horse, mouse, rat and guinea pig.

15. The method of claim 14, wherein said animal is a germ free, gnotobiotic, specific pathogen free or transgenic animal.

16. A method of producing a product of interest comprising isolating said product from the feces of the animal according to any of claims 1-6, wherein said modified bacteria is capable of producing said product.

17. The method of claim 16 wherein said animal is selected from the group consisting of a cow, pig, goat, sheep, rabbit, horse, mouse, rat and guinea pig.

18. A method of treating an animal with a gastroenterological disease comprising administering to an animal in need of such treatment an effective amount of a formulation comprising a modified bacteria capable of producing a therapeutic agent.

19. A method of inducing an immune response specific to a foreign antigen in an animal comprising administering to the digestive tract of the animal a composition comprising a modified bacteria capable of producing said foreign antigen.
